(19) United States
(12) Patent Application Publication
(10) Pub. No.: US 2005/0180922 A1
(43) Pub. Date: Aug. 18, 2005

(54) Block co-polymer worm micelles and methods of use therefor

(76) Inventors: Dennis E. Discher, Philadelphia, PA (US); Paul Dalhaimer, Sheboygan, WI (US)

Correspondence Address: Evelyn H. McConathy;DRINKER BIDDLE & REATH LLP, One Logan Square, 18th & Cherry Sts, Philadelphia, PA, 19103, US

(21) Appl. No.: 10/913,660

(22) Filed: Aug. 6, 2004

Related U.S. Application Data
(60) Provisional application No. 60/544,363, filed on Feb. 12, 2004.

(51) Int. Cl. A61K 48/00; A61K 49/00; A61K 9/127

(52) U.S. Cl. 424/9.52; 424/450; 514/44; 514/2

(57) ABSTRACT

Provided are worm-like micelles, capable of encapsulating at least one encapsulant, wherein each worm-like micelle comprises one or more wholly synthetic, polymeric, super-amphiphilic molecules that self assemble in aqueous solution, without organic solvent or post assembly polymerization; and wherein at least one of said super-amphiphilic molecules is a hydrophilic block copolymer, the weight fraction (w) of which, relative to total copolymer molecular weight, directs assembly of the amphiphilic molecules into the worm-like micelle of up to one or more microns in length, and determines its stability, flexibility and convective responsiveness. Also provide are methods of preparing and methods of using the worm-like micelles, particularly when loaded with one or more encapsulants. The loaded worm-like micelles of the present invention are particularly suited for the stable and controlled transport, delivery and storage of materials, either in vivo or in vitro.

### CROSS REFERENCE TO RELATED APPLICATIONS

This application claims priority to U.S. Provisional Application No. 60/544,363, filed Feb. 12, 2004, the content of which is herein incorporated in its entirety.

### GOVERNMENT SUPPORT

This work was supported in part by grants from the National Science Foundation, grant number NSF-MRSEC, and also by grants from the National Institutes of Health, grant number NIH R21. The government may have certain rights in this invention.

### FIELD OF THE INVENTION

The present invention relates to the preparation and characterization of worm-like micelles formed from block copolymer amphiphiles, and their use as delivery vehicles, particularly when cell-targeted.

### BACKGROUND OF THE INVENTION

Biomembrane stability and other thermo-mechanical properties of the cell have been applied in phospholipid vesicles (liposomes) that have been assembled in vitro to effectively encapsulate and deliver a long list of bioactive agents (Needham et al., in Vesicles, M. Rosoff, Ed., Dekker, New York, 1996, chap. 9; Cevc & Lasic in Handbook of Biological Physics, 1995, chaps. 9-10; Koltover et al., Science 281:78 (1998); Harasym et al., Cancer Chemother. Pharinacol. 40:309 (1997)). Functionally targeted assemblies have been of particular interest for diagnostic and therapeutic applications.

The typical liposome comprises one or more bilayer membranes, each approximately 5 nm thick, composed of amphiphiles, such as phospholipids. Each bilayer exists as a temperature- and solvent-dependent lamellar phase that is, at its surface, in a liquid, gel, or liquid-gel coexisting state. Small amphiphiles of natural origin have inspired the engineering of high molecular weight analogs, which also self-assemble in aqueous solution into complex phases in aqueous media similar to those observed for phospholipids (Uchegbu et al, J. Pharm. & Pharmacol. 50:453-458 (1998)).

Spherical shell structures smaller than a few hundred nanometers, and requiring the presence of organic solvents mixed into water to drive their formation, include those assembled from various block copolymers as observed by Yu et al., Macromolecules 31:1144-1154 (1998); Ding et al., J. Phys. Chem. B 102:6107-6113 (1998); Henselwood et al., Macromolecules 31:4213-4217 (1998). Many wholly synthetic, amphiphilic molecules are significantly larger (in molecular weight, volume, and linear dimension) than phospholipid amphiphiles, and have therefore been called “super-amphiphiles” (Cornelissen et al., Science 280:1427-1430 (1998)). Cornelissen et al. used polystyrene (PS) as a hydrophobic fraction in a series of synthetic block copolymers designated PS40-b-(isocyano-L-alanine-L-alanine) to produce small collapsed vesicles with diameters ranging from tens to hundreds of nanometers, and a bilayer thickness of 16 nanometers.

Amphiphilic multiblock copolymers self-assemble in water into various stable morphologies. Synthetic control over the molecular composition permits novel controls over the properties of membranes assembled from super-amphiphiles (Hajduk et al., J. Phys. Chem. B 102:4269-4276 (1998)). For instance, a super-amphiphilic polymer can be made far more reactive than a much smaller phospholipid molecule simply because more reactive groups can be designed into the polymer. Both amphiphiles and super-amphiphiles can exist in a broad variety of microphases and bulk phases that include not only lamellar, but also hexagonal, cubic, and more exotic phases (see review by Lipowsky and Sackmann, in Handbook of Biological Physics, 1995, chap. 8; Bates, Science 251:898-905 (1991)).

To control drug release from delivery systems based on chemically active monomers, such as phospholipase sensitive lipids (Jorgensen et al., FEBS Lett. 531:23-27 (2002); Davidsen et al., Biochim. Biophys. Acta 1609:95-101 (2003)), methods have included destabilization by pH or light (Gerasimov et al., Biochim. Biophys. 1324:200-214 (1997); Wymer et al., Bioconjugate Chemistry 9:305-308 (1998); Boomer et al., Chemistry and Physics of Lipids 99:145-153 (1999); Adlakha-Hutcheon et al., Nature Biotechnology 17:775-779 (1999)), and the introduction of polyethyleneglycol (PEG) (Kirpotin et al., FEBS Lett. 388:115-118 (1996); Zalipsky et al., Bioconjugate Chemistry 10:703-707 (1999); Shin et al., J. Controlled Release 91:187-200 (2003); Boomer et al., Langmuir 19:6408-6415 (2003); Bergstrand et al., Biophysical Chemistry 104:361-379 (2003)). Originally added as stabilizers, a small percentage (5-10%) of PEG-lipid was found to also delay liposome clearance (Klibanov et al., FEBS Lett. 268:235-237 (1990); Photos et al., J. Controlled Release 90:323 (2003)).

Diblock copolymers are prepared, for example, using a two-step anionic polymerization procedure (Hillmyer et al., Macromolecules 29:6994-7002 (1996), wherein copolymers are dissolved in chloroform and dried on glass to form a film that is hydrated with water at 50-60° C. In dilute aqueous solutions certain diblock copolymers, such as polyethyleneoxide-polyethylethylene (PEO-PEE, also referred to simply as OE, wherein PEO is structurally equivalent to PEG), have been shown to form unilamellar vesciles and micelles in which polyethyleneoxide-polybutadiene (PEO-PBD, also referred to simply as OB) mesophases were successfully cross-linked into bulk materials with completely different properties, notably an enhanced shear elasticity (Won et al., Science 283:960-963 (1999); Discher et al., Science 284:1143-1146 (1999)). The resulting microstructures, termed polymersomes, though assembled in water, can withstand dehydration, as well as exposure to organic solvents, such as chloroform (U.S. patent appl. Ser. No. 09/460,605), and controlled release of encapsulants from such vesicles was subject to denaturaturation of the selected blend of block copolymers (Ahmed et al., J. Controlled Release, 96:37-53 (2004); CIP of U.S. patent appl. Ser. No. 09/460,605, based upon Provisional Appl. 60/459,049).

Block copolymers of both PEG and a hydrolytically susceptible polyester of either polylactic acid (PLA) (Belbella et al., Internat'l J. Pharmaceutics 129:95-102 (1996); Anderson et al., Adv. Drug Delivery Rev. 28:5-24 (1997); Brunner et al., Pharmaceutical Research 16:847-853 (1999); Woo et al., J. Controlled Release 75:307-315 (2001)) or polycaprolactone (PCL) (Pitt in Biodegradable Polymers as Drug Delivery Systems, Langer, Chasin (eds.), Marcel Dekker, New York, N.Y., 1990, pp. 71-120; Chawla et al., Internat'l J. Pharmaceutics 249:127-138 (2002)) have been described (Matsumoto et al., Internat'l J. Pharmaceutics 185:93-101 (1999); Allen et al., J. Controlled Release 63:275-286 (2000); Panagi et al., Internat'l J. Pharmaceutics 221:143-152 (2001); Riley et al., Langinuir 17:3168-3174 (2001); Avgoustakis et al., J. Controlled Release 79:123-135 (2002); Discher et al., Science 297:967-973 (2002a); Meng et al., Macromolecules 36:3004-3006 (2003); Ahmed et al., Langmuir 19:6505-6511 (2003)). Vesicle formulations prepared using hydrolyzable diblock copolymers of polyethyleneglycol-poly-L-lactic acid (PEG-PLA) or polyethyleneglycol-polycaprolactone (PEG-PCL), with or without inert PEG-PBD (Discher et al., supra, 1999)), have been shown to provide programmed control over release kinetics (Ahmed et al., supra, 2004; CIP Patent Appl., supra), based on the general principle of blending degradable and inert copolymers.

Controlled release drug-delivery vehicles run the gamut from self-assemblies of lipids (liposomes) (Gref et al., Science 263:1600-1603 (1994); Lasic et al., Curr. Op. Solid St. M. 3:392 (1996)) to biochemically modified quantum dots (Akerman et al., Proc. Nat'l Acad. Sci. (USA) 99:12617-12621 (2002)). However, all vehicles studied to date have had the same spherical geometry. Spherical liposomes (diameter ˜100 nm) are cleared from the vasculature of small mammals hours after injection (Blume et al., Biochim. Biophys. Acta 1029:91 (1990)), although the polymersomes, assembled from PEG-based copolymers, have shown an increased circulation time compared to liposomes, wherein polymersomes have half-lives of approximately one day in vivo (Photos et al., supra, 2003).

Encapsulation studies have shown loading in the controlled release vesicles to be comparable to liposomes. Rates of release of encapsulants from the hydrolysable vesicles were accelerated by an increased proportion of PEG, but were delayed with a more hydrophobic chain chemistry, i.e., PCL. Rates of release rose linearly with the molar ratio of degradable copolymer blended into membranes of a non-degradable, PEG-based block copolymer (PEG-polybutadiene). Thus, poration occurred as the hydrophobic PLA or PCL block was hydrolytically scissioned, progressively generating an increasing number of pore-preferring copolymers in the membrane, which when combined with the phase behavior of the amphiphiles, triggered transition from membrane to micelle kinetics, resulting in controlled release of the encapsulant.

Worm micelles have been formed from small (˜500-1000 g/mol) amphiphiles (Walker, Curr. Opin. Colloid Interface Sci. 6:451 (2001)), but were unstable and quickly fell apart in dilute aqueous concentrations. As a result, typical surfactant worm micelles could not survive injection as intact aggregates into the circulation of an animal. Nevertheless, although past studies of lipid and surfactant-based worm micelles have been frustrated by the low stability of the assemblies, other cylindrically shaped delivery systems occur in nature in the form of filamentous phages, which have been studied for therapeutic applications. For example, Ruoslahti and coworkers used the micron-long filamentous phage, M13, in a phage display method for identifying ligands that bind to xenoplants of various human cancers (Pasqualini et al., Nature 380:364-366 (1996); Pasqualini et al., Nat. Biotechnol. 15:542-546 (1997)). Once the targeting ligand was identified, it was chemically conjugated to a chemotherapeutic hydrophilic drug (doxorubicin) and successfully used to treat tumors in live animals (Arap et al., Science 279:377 (1998)).

Therefore, in light of the success of filamentous phage and polymersome delivery systems, there has clearly been a need for novel, stable, aqueously-formed constructs, which can be broadly engineered, but still have the advantageous features of worm micelles necessary to permit biological delivery, including: biocompatibility, selective permeability to solutes, the ability to retain internal aqueous components and control their release, and the ability to deform while remaining relatively tough and resilient. Moreover, such novel constructs must also be able to target selected cells or cell types for delivery of encapsulated contents.

### SUMMARY OF THE INVENTION

The present invention meets the need in the art by providing worm micelles as delivery vehicles, particularly drug delivery vehicles, that are prepared from high molecular weight diblock amphiphilic copolymers (e.g., >1-4000 g/mol), which in contrast to early worms prepared from low molecular weight lipids and surfactants, are stable, synthetic, non-living assemblies, even at body temperature (37° C.). The preferred copolymers comprise a hydrophilic PEO (polyethylene oxide) block and one of several hydrophobic blocks that drive self-assembly of worm-like micelles, up to microns in length, in water and other aqueous media. The PEO block of the polymer (which is the same as polyethylene-glycol; PEG) is widely known to make interfaces very biocompatible, thus the worm-like micelles are stable in blood in vitro and in blood flow in vitro and in vivo.

Visualization of the worm-like micelles can be achieved by fluorescence microscopy after incorporating fluorescent dyes into the micelle cores dyes. Increasing the molecular weight of the copolymers increases both the diameter of the worm-like micelles (from about 10 to 40 nm) and their stiffness. In addition, in the present invention, biotinylated copolymers were blended with pristine copolymers prior to forming micelles by simple hydration of a dried copolymer film.

For drug delivery, the worm micelles of the present invention are shown to be able to incorporate a range of hydrophobic drugs into the cores of the worm-like micelles, and methods are provided to chemically modify the ends of the PEO blocks to make the worm-like micelles specifically bind to suitable surfaces and cells. The present invention, therefore, provides worm micelles which encapsulate one or more “active agents,” which include, without limitation compositions such as a drug, therapeutic compound, dye, nutrient, sugar, vitamin, protein or protein fragment, salt, electrolyte, gene or gene fragment, product of genetic engineering, steroid, adjuvant, biosealant, gas, ferrofluid, or liquid crystal. The thus “loaded” worm micelle may be further used to transport an encapsulatable material (an “encapsulant”) to or from its immediately surrounding environment.

The present invention provides methods of using the worm micelles to transport one or more of the above identified compositions to or from a patient in need of such transport activity. For example, the worm micelle could be used to deliver a drug or therapeutic composition to a patient's tissue or blood stream, or it could be used to remove a toxic composition from the blood stream of a patient with, for example, a life threatening hormone or enzyme imbalance.

Also provided by the present invention are methods of preparing an “empty” worm micelle, wherein the preferred methods of preparation include at least one step consisting of a film rehydrating step, a bulk rehydrating step, or an electroforming step.

Further provided are methods for controlling the release of an encapsulated material from a worm micelle. For example, the worm-like micelles can be fragmented to sub-micron lengths, if desired, and they will flow through nanoporous matrices, including recognized models for brain tissue matrix. Based upon findings using the cytotoxic drug paclitaxel commonly used against cancer cells, further provided is a method of using the worm-like micelles of the present invention to efficiently target and kill cells.

Thus, it is an object of the invention to provide worm micelles for use as drug delivery vehicles, as well as methods for their preparation and for the encapsulation of one or more active agents, and for the controlled release of same. It should be noted that the terms “worm micelle” and “worm-like micelle” are used interchangeably herein to mean the same assembly, and are often simply referred to as “worms” or “micelles.”

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIGS. 1a and 1b depict worm-like micelles formed from the diblock copolymers PEO-PEE and PEO-PBD. FIG. 1a schematically illustrates a worm with d≈14 nm core. Spheres represent fluorescent dye molecules. The inset distribution of measurable contour lengths, L, is fit by Freq=L exp(−L/L*), where L*=√(C^{α}) with C˜1 mg of copolymer/mL of H₂O at the time of worm formation, and α is the energy driving self-assembly. FIG. 1b photographically shows a progression of pristine worm in a suspension of blood cells. For several seconds, the worm is elongated by a flow of v≈10 μm/s (I) before being sequestered in a stagnant zone, where the worm simply diffuses (II and III). Thus, the worms clearly do not stick to the cells. The reference cell (*) is stuck to the coverslip. Scale bar is 10 μm.

FIG. 2 schematically illustrates persistence lengths of pristine PEO-PBD and PEO-PEE worm-like micelles (labeled open circles), plus selected cross-linked PEO-PBD blends (circles with X-mark) (details in Table 1, below) in comparison to ubiquitously expressed biopolymers (filled squares). Inset shows formula for calculating l_{P} for worms stiffer than OB9 through vectors tangent to the worm backbone separated by distance s.

FIG. 3 schematically illustrates scaling relationship of worm-like micelle diameter (d) as function of the number of hydrophobic groups (Nₕ) in OE (filled squares) and OB (open squares) class copolymers in Table 1, below. Diameters were measured from cryo-TEM images of the worms. The inset cryo-TEM image shows an OB 18 worm-like micelle and the cross-sectional intensity profile used to estimate its diameter, d=27 nm.

FIG. 4 graphically shows the calculated stiffness in terms of persistence lengths l_{P} of worms as a function of varying hydrophobic core diameters (d). Diameters are calculated from Cryo-TEM images and persistence lengths are calculated from fluorescence video microscopy for seven different worm forming diblock copolymers listed in Table 1, below. Inset images show fluorescence snapshots and of three different pristine worm-like micelles of varying stiffness worms, representative of small, medium, and large d values, freely diffusing in a pseudo-2D chamber along with backbone traces taken every ˜0.5 sec. Scale bars are 5 μm.

FIGS. 5a-5e schematically illustrate backbone traces of chemically cross-linked OB3/OE6-blended worm-like micelles showing the spontaneous curvature of worms just above and below the percolation point (X_{OB3}˜0.15-0.2). In FIG. 5a the worm is below the percolation point; FIGS. 5b and 5c show the effect of increasing the amount of cross-linked PEO-PBD through the percolation mole fraction, increasingly solidifying the worm backbone, until in FIGS. 5d and 5e, it finally exhibits only a rigid body rotation about an easily identifiable, spontaneously curved conformation. Each trace is taken about every 0.3 seconds.

FIGS. 6a-6d show snapshots (inversely intensified) and backbone traces of pristine and completely cross-linked worm-like micelles. Worms are confined between two glass coverslips providing a pseudo-two dimensional geometry. In FIG. 6a, the pristine worm explores much of the available configuration space (l_{P}≈0.5 μm), τ_{R/L}≈2.6 seconds; 15 backbone traces are overlaid. In FIG. 6b, a chemically cross-linked worm displays dynamics that are similar to rigid body rotation (l_{P}≈115 μm), τ_{R/L}≈0.2 seconds; 10 backbone traces are overlaid. Scale bars are 5 μm. FIG. 6c graphically shows representative exponential fits of autocorrelation function decays in R/L vs time for pristine (FIG. 6a) and fully cross-linked worms (FIG. 6b), highlighting the disparate dynamics between the two systems. FIG. 2d plots R/L as a function of time for fully cross-linked worms, showing that the rodlike worms are ergodic objects at the depicted time scales.

FIG. 7 graphically shows worm micelle dynamics and stiffness measures as a function of cross-linking. Worms with cross-linked OB3 in OE6 (OB3 mole fraction, X) below the percolation point X_{C} have very similar relaxation times, τ_{R/L}, for normalized end-to-end length, R/L; whereas worms above X_{C} have decreasing τ_{R/L}. Thus, the effective stiffness of the partially cross-linked worms is given by the fluctuations in R/L. For X<X_{C}, worms have very similar stiffness; whereas for X>X_{C}, worm stiffness linearly increases.

FIG. 8 shows the stability of biotinylated worm micelles. Plot shows average contour length of biotinylated (25%) worm micelles, as measured by fluorescence microscopy after irreversible binding to an avidin-coated surface. Scale bar is 5 μm.

FIGS. 9a-9c photographically depicts the effect of mild sonication to systematically reduce worm micelle contour length. FIG. 9a shows OEX-biotin worm micelles adsorbed to a 1% (mol) avidin in BSA coated surface, wherein many worms are longer than 5 μm.

FIG. 9b shows the same OEX-biotin sample after 2 minutes of sonication, wherein most worms are less than 5 μm. FIG. 9c shows an AFM image of worm micelles after 5 minutes of sonication revealing sub-micron length worms. Scale bar is 5 μm.

FIGS. 10a-10e photographically and graphically show the preferential binding of biotinylated worm micelles to smooth muscle cells. Worm micelles containing an orange PKH26 hydrophobic dye were incubated in cultures of smooth muscle cells for 5 hours. FIG. 10a shows biotinylated worms (OEX-biotin). Scale bar is 20 um. FIG. 10b shows pristine worms (OEX). FIG. 10c shows ‘No addition,’ wherein ‘N’ denotes nucleus. FIG. 10d shows the effect of free PKH26 dye added to smooth muscle cells in culture, and shows that the destination of the dye is the same, whether free or encapsulated in biotinylated worm micelles. FIG. 10e graphically represents intensity measurements on at least 10 cells, showing that cells incubated with biotinylated worms appear on average 10-fold brighter than cells incubated with equal concentration of pristine worms.

FIGS. 11a-11c photographically and graphically show that worm micelles of modified OE (OEX) stay in circulation longer than 100 nm vesicles. FIG. 11a provides a series of snapshots of worm micelles taken from circulation over the course of 6 days as shown. Scale is 20 μm; inset scale bars are 2 μm. FIG. 11b shows intensity (mass) of fluorescently-labeled assemblies versus time after tail-vein injection into rat models, and demonstrate that worm micelles stay in circulation much longer than spherical assemblies, but begin to be cleared after 3 days. FIG. 11c graphically displays decay in contour length over the same time period as represented in FIG. 11b, and show that while long worms fragment, short worm micelles are cleared more rapidly than longer worms.

FIG. 12 photographically depicts a fluorescence image of worm micelles loaded with Oregon green taxol in a molar ratio to copolymer of 1:14. Worms are seen to freely diffuse in a narrow gap chamber. Scale bar as shown is 5 μm.

FIGS. 13a and 13b photographically and graphically show that worm micelles functionalized with THRPPMWSPVWP, and loaded with Oregon Green taxol, preferentially bind to CEF-hTfR cells. FIG. 13a shows two photographs (FIG. 13a-left and 13a-right) demonstrating the effect of incubating CEF cells and CEF-hTfR cells, respectively, with functionalized worms for 4 hours. FIG. 13b graphically provides an hTfR saturation curve, showing non-specific binding of worms to CEF cells when the concentration of worms is high.

### DETAILED DESCRIPTION OF PREFERRED EMBODIMENTS OF THE INVENTION

The present invention characterizes giant and stable worm-like micelles formed in water from a series of blended and cross-linkable polyethyleneoxide-based (PEO) diblock copolymer amphiphiles that mimic the flexibility of various cytoskeletal filaments, and provide methods for encapsulation and cell-targeted micropore drug delivery. Worm micelles are amphiphilic aggregates poised in size between molecular scale spherical micelles and much larger lamellar structures, such as vesicles, and fluidity and hydrodynamics play important roles in there formation.

The worm micelles of the present invention are formed from synthetic, amphiphilic copolymers. An “amphiphilic” substance is one containing both polar (water-soluble) and hydrophobic (water-insoluble) groups, and polymers are macromolecules comprising connected monomeric units. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains).

The preferred class of polymer selected to prepare the worm micelles of the present invention is the “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

In the “melt” (pure polymer), a diblock copolymer may form complex structures as dictated by the interaction between the chemical identities in each segment and the molecular weight. The interaction between chemical groups in each block is given by the mixing parameter or Flory interaction parameter, _{χ}, which provides a measure of the energetic cost of placing a monomer of A next to a monomer of B. Generally, the segregation of polymers into different ordered structures in the melt is controlled by the magnitude of _{χ}N, where N is proportional to molecular weight. For example, the tendency to form lamellar phases with block copolymers in the melt increases as _{χ}N increases above a threshold value of approximately 10.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material, producing the numerous resulting structural phases.

To form a stable membrane in water, the absolute minimum requisite molecular weight for an amphiphile must exceed that of methanol HOCH₃, which is undoubtedly the smallest canonical amphiphile, with one end polar (HO—) and the other end hydrophobic (—CH₃). Formation of a stable lamellar phase more precisely requires an amphiphile with a hydrophilic group whose projected area, when viewed along the membrane's normal, is approximately equal to the volume divided by the maximum dimension of the hydrophobic portion of the amphiphile. Therefore, assembly of diblock copolymer amphiphiles into one of the worm micelles depends primarily on the weight fraction (w) of the hydrophilic block relative to the total copolymer molecular weight (Discher et al., supra, 1999; Hajduk et al., supra, 1998; Zhang et al., Science 268:1728 (1995)). Higher w gives predominantly spherical micelles, whereas lower w yields vesicles (Israelachvili, In Intermolecular & Surface Forces, Academic Press: London, 1992, pp 380-382).

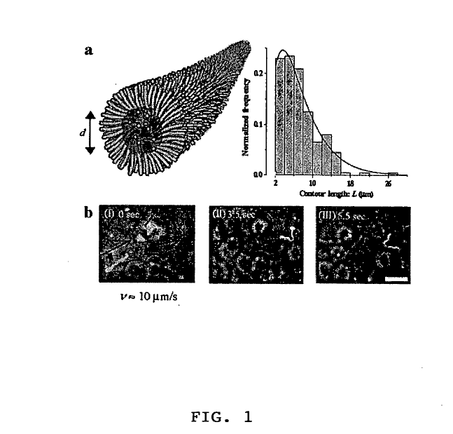

For example, using a diblock copolymer comprising a hydrophilic polyethyleneoxide (PEO) block and a hydrocarbon-based hydrophobic block, spontaneously form into vesicles or polymersomes when the bulk copolymer is added to water when the fraction of the PEO block is ˜25-42%. However, with a relatively small increase in the PEO fraction, such that W_{EO}≈45-55%, hydration of the PEO overcompensates, and osmotic force induces a curvature in the aggregate, leading to the assembly of mainly worm micelles (FIG. 1a). Such aggregate formation is strongly driven by the relatively high molecular weight of the hydrocarbon segment. This creates an interfacial tension, which separates the core from PEO, as well as the bulk aqueous phase. Despite this sensitivity in composition, the worm micelle assemblies of 4000-5000 g/mol diblocks prove exceedingly stable, yet flexible.

Cryo-transmission electron microscopy (cryo-TEM) has shown that the worm micelles, made of block copolymers of molecular weight MW ˜4 kDa, have hydrophobic cores of ˜10 nm. Worm micelles assembled from PEO-based nonionic copolymers prove extremely stable in aqueous media, and the dynamics of the worms can be directly visualized with fluorescence microscopy techniques (FIG. 1b). By blending and polymerizing inert (OE6) and cross-linkable (OB3) copolymers, micelles up to tens of microns long (or longer) are formed with sub-micron persistence lengths (l_{P}) that continuously span more than 2 orders of magnitude —from submicron (about 500 nm) to submillimeter (about 100 μm), in agreement with estimations from neutron scattering (Won et al., J. Phys. Chem. B 105:8302 (2001)). Under quiescent conditions, the persistence length of the pristine worms is just large enough for the backbone to be clearly visualized when the worm is confined in a gap or a microcapillary.

Although the diameter (d) of the worm micelles is similar to the membrane thickness of polymersomes, the Brownian dynamics of worm micelles are highly pronounced in contrast to the membranes. Autocorrelation analyses of the easily identified end-to-end distance yields relaxation times of seconds for the micron long worms. Although these are fluid assemblies, the stability of the worms is clear, however, and appears fully consistent with the high γ that drives membrane formation and underlies the stability of the micelles.

In a flow field, rather than under quiescent conditions, the fluid worms orient and stretch with DNA-like scaling, and respond in a way roughly in agreement with present theory for polymers under flow (see Example 1). When viewed by microscope, the worms stuck occasionally at a single point to a coverslip. This was surprising, given that the worms themselves are fluid. One would expect that under the imposed flow, the worms should slip, rather than stick. However, the worms increasingly straighten under high flow, but their thermally accessed conformational space is always narrowest near the point of attachment. At the free end, these trumpet shapes extend up away from the coverslip, but project down into an ellipse of fluctuations that is clearly much larger than the microscopic point of attachment. It is also dependent on the mean flow velocity, as well as the length of the worm. Nevertheless, it is clear, however, that the worms can withstand very high flow fields which can be estimated to impose tensions <1 μN/m (see calculation from a plane Poiseuille flow model in Example 1; N=Newton).

Worm Flexibility. The two types of worms—fluid or cross-linked—represent the two extremes at either end of a continuous stiffness scale that can be experimentally realized by blending saturated polyethylethylene (PEE) copolymer with the cross-linkable polybutadiene (PBD) copolymer. The two copolymers have already been shown (using membranes) to be fully miscible, and the PBD can be successfully reacted to give a range of stabilities and stiffnesses (Discher et al., supra, 2002a). For worms made with similar MW copolymers, there appears an interesting percolation of the rigidity at relatively low mole fractions of PBD (˜20%). For worms, this represents a quasi-one-dimensional fluid-to-solid transition with potentially interesting dynamics of worms blended at or near the transition point.

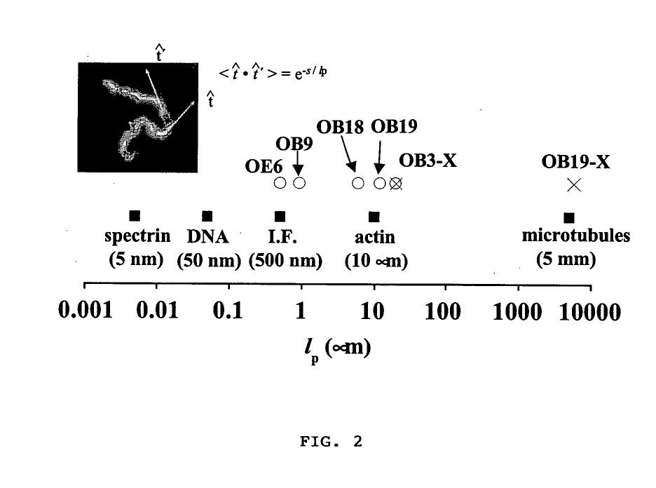

In a comparison to cytoskeletal filament rigidities, FIG. 2 summarizes the cross-linking result above, and adds to it the measured persistence lengths for giant worm-like micelles formed from a broader, representative subset of copolymers in Table 1. The inset in FIG. 2 shows the formula for calculating l_{P} for worms stiffer than OB9 through vectors tangent to the worm backbone separated by distance, s (see, Grosberg et al., Statistical Physics of Macromolecules, AIP Press, Woodbury, N.Y., 1994, Chap. 1). For worms with flexibility equal to or greater than OB9, l_{P} is calculated using <R²>=2l_{P}L[1−e^{-L/l}^{ₚ})] where R is the end-to-end distance of the worm and L is the contour length. As schematically show OB class worms can be pristine or fully cross-linked through polymerization, and OE diblocks can be added to dilute the cross-links and decrease worm stiffness. OB19 worms have diameters that are ˜2.5 times larger than OB3 worms. Therefore, from the beam theory, fully cross-linked OB19 worms would be 50 times stiffer (d_{OB19}/d_{OB3}) (Comelissen et al., supra, 1998) than a fully cross-linked OB3 worm (l_{P}˜115 μm). While not easily measured, this would mean l_{P}˜6 mm, which rivals the stiffness of a microtubule. As described in greater detail below, the value for l_{P} is obtained from a tangent-tangent correlation function for OB18 and OB19 worms and an integrated version of the tangent-tangent correlation function involving fluctuations in the end-to-end length for all other, more flexible worms.

As shown, worm-like micelles can emulate the bending rigidity of various ubiquitous biopolymers, from intermediate filaments to microtubules, through selection of different sized copolymers and chemical fixation of unsaturated butadiene bonds. While the principles behind blending and cross-linking are increasingly understood, the subtlety in controlling rigidity with worm diameter stems from the hypothesis that molecules in a fluid worm will rearrange and significantly relax any curvature stress. Factors affecting worm stiffness include scaling of l_{P} with worm diameter d, as well as worm branching and spontaneous curvature effects in cross-linking.

There are presently at least two ways to explore for augmenting the bending rigidity of worm micelles given the present chemistry: (1) chemically cross-link the BD blocks in the worm core to create a solid worm-like micelle, and/or (2) increase the diameter of the worm by assembling the worm from larger copolymers. In the first instance, double bonds in the hydrophobic block of PBD allow cross-linking to be introduced by solution free radical polymerization into the worm cores (Won et al., supra, 1999). Worms can thus be made even more stable and solid, emulating a classic covalent polymer chain, but at a more mesoscopic scale. Such cross-linked worms often appear kinked. This occurs simply because the polymerization within the core is done while the worm is flexing in solution. When stuck at a point and examined under flow, such fully cross-linked worms generally just wobble and pivot about the attachment, with any kink remaining locked in and nearly unperturbed by the flow-imposed stresses.

Free radical cross-linking within OB3 worms increases worm persistence length by more than 100-fold from l_{P}=0.5 μm to a cross-linked value, l_{PX}˜100 μm. As used herein, a term followed by an “X” means that it is fully cross-linked, e.g., OB3-X is fully cross-linked OB3. The persistent length of a cross-linked species is referred to as l_{PX}. Moreover, to interpolate both within this range of rigidities and also from fluid to solid states, a PEO-PEE analog of OB3 (OE6) can be blended into the worm in varying concentrations before free radical polymerization of the PBD double bonds.

By combining techniques (1) and (2) above, OB worms of large diameter (up to d=39 nm; Table 1) can be fully cross-linked to form almost inflexible solid cylinders (OB19-X) with a persistence length approaching that of a microtubule. The copolymers listed in Table 1 thus span bending rigidities of ubiquitously expressed biopolymers that range from intermediate filaments to microtubules.

TABLE 1
Structural Details of Poly(ethylene oxide)-Polybutadiene (OB) and
Poly(ethylene oxide)-Polyethylene (OE) Diblock Copolymers.

| Designation | Polymer formula | Mₙ (kg/mol) | w_{EO} | d(nm)* |
|---|---|---|---|---|
| OE2 | EO₄₄-EE₃₇ | 3.6 | 0.54 | 10.8* |
| OE6 | EO₄₆-EE₃₇ | 4.1 | 0.48 | 12.5* |
| OE7 | EO₄₆-EE₃₇ | 3.9 | 0.45 | 11.4^{†} |
| OB3 | EO₅₅-BD₄₅ | 4.9 | 0.51 | 14.2^{‡} |
| OB9 | EO₅₀-BD₅₄ | 5.2 | 0.43 | 15.7 |
| OB18 | EO₈₀-BD₁₂₅ | 10.4 | 0.35 | 27.0 |
| — | EO₁₀₅-BD₁₇₀ | 14 | 0.34 | 34.0 |
| OB19 | EO₁₅₀-BD₂₅₀ | 20 | 0.33 | 39.0 |

Diameters denoted by (*) were determined by a best-fit of referenced and measured data.
^{†}see, Won et al., J. Phys. Chem. B 106:3354 (2002);
^{‡}see, Won et al., supra, 1999.
⁺see, Jain and Bates, Science 300:460-464 (2003).

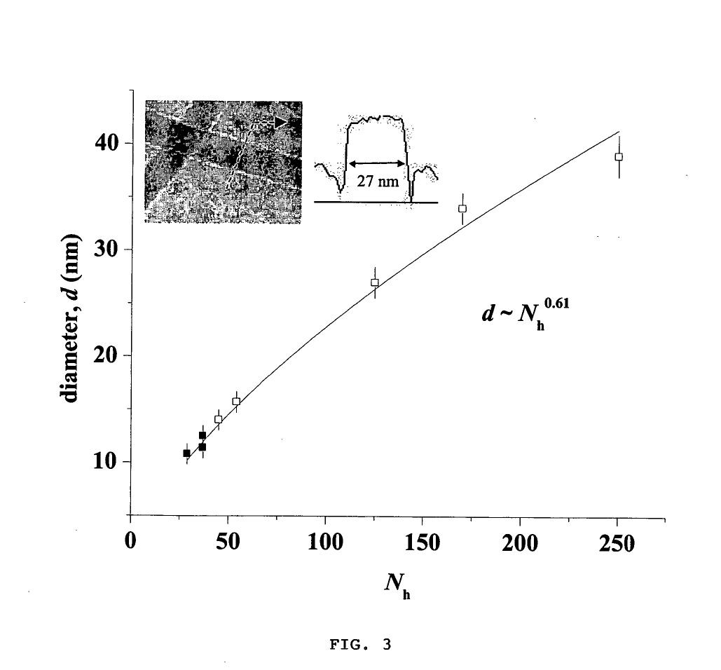

Measurements of worm diameter d from cryo-TEM images show a systematic dependence on the length of the hydrophobic chain (Nₕ), which has also been found for membranes (Bermudez et al., Macromolecules 35:8203-8208 (2002)). Fitting a power law to the referenced and measured data in Table 1, produces the curve shown in FIG. 3, wherein the diameter of the worms fits best to d=1.38Nₕ⁰⁶¹. A fully stretched polymer of Nₕ groups would theoretically assemble into an object with diameter, d˜Nₕ¹, whereas ideal random coils, such as in a melt, would give an object with d˜Nₕ^{0.5}. The copolymers studied herein are in the strong segregation limit (SSL) where interfacial tension, γ, balances chain entropy, so that d˜Nₕ^{0.67} is expected (Bates, supra, 1991; Bermudez et al., supra, 2002). The scaling exponent obtained of 0.61 is thus slightly closer to the SSL expectations than the scaling found for membranes assembled from a subset of the same copolymers in Table 1.

Moreover, the radius of gyration (Rg) can be calculated using Rg=b(Nₕ/6), where b=0.54 nm has been experimentally determined for the PEO-PEE copolymers (Almdal et al., Macromolecules 35:7685 (2002)). For OE7, for example, Rg=1.3 nm, which indicates that the copolymer is stretched about 4- to 5-fold compared to the worm radices, d/2 (Table 1). This result is fully consistent with strong lateral squeezing of chain configurations by interfacial tension that extends the chain into the core and thus forms the basis for SSL theory. Thus, while scaling of d with Nₕ alone (d˜Nₕ^{0.61}) is less convincing of SSL versus a simpler melt (d˜Nₕ^{0.5}), the strong stretching is indicative of the SSL.

Given the wide range of core diameters, d, for the worms in Table 1, the scaling relation for the worm persistence length, l_{P}, can be experimentally determined. Dimensional analysis shows that a fluid cylinder whose rigidity is dominated by y has a persistence length that scales with core diameter in the form l_{P}=φγd³/k_{B}T, where φ is a constant. Based on extensive measurements of membrane elasticity, γ is already known to be a single constant for the OB and OE series of copolymers (Bermudez et al., supra, 2002). Conversely, a solid rod or cylinder, also of diameter d, follows the classical beam theory scaling of l_{P}˜d (Comelissen et al., supra, 1998; Landau et al., Theory of Elasticity, 3^{rd} ed., Butterworth-Heinemann: Oxford, 1986, chap. 2), where the energy scale for a beam is set by an elastic constant (E) for the core, rather than by γ.

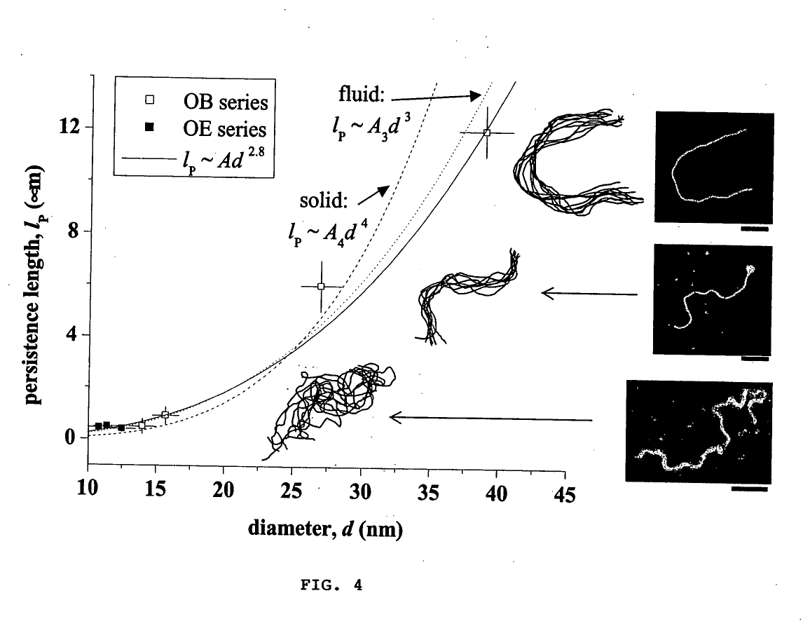

FIG. 4 shows the calculated persistence lengths of worms of varying hydrophobic core diameters along with fluorescence snapshots and backbone traces of three worms representative of small, medium, and large d values. When fit by power laws (best fit), the data shows that bending rigidities of these worms have a scaling exponent of 2.8 (l_{P}˜d^{2.8}). Despite chain entanglement in the core, which could effectively solidify it, the scaling result more closely follows the cubic scaling behavior of classical fluid assemblies of lipid-size amphiphiles (˜d3), rather than solid-core cylinders, rods or beams (˜d 4) (A=0.0004, A₃=0.00023, A₄=9×10⁻⁶). Therefore, given this exponent and γ=25 pN/nm, then φ= 1/20 in l_{P}=φγd^{2.8}/k_{B}T. As a result, by polymerizing the unsaturated bonds of assembled copolymers, fluid worms are clearly converted to solid-core worms, extending the bending rigidity from that of intermediate filament biopolymers to actin filaments and, in principle, microtubules. Remarkably, this result is similar to lipid assemblies with φ= 1/30 characteristic of semi-coupled monolayers (Bloom et al., Q. Rev. Biophys. 24(3):293-397 (1991)).

Cross-linking percolation and spontaneous curvature. As noted above, cross-linked blends of PEO-PBD and PEO-PEE copolymers form worms that span bending rigidities between the fluid PEO-PEE worm (or pristine PEO-PBD worm) and the fully cross-linked, solid PEO-PBD worm. Through partial cross-linking, polymerized worms are further shown to lock in spontaneous curvature at a novel fluid-to-solid percolation point.

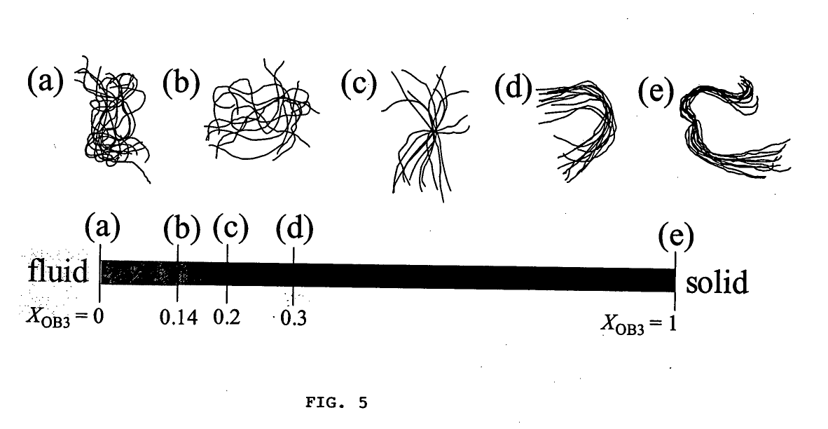

A percolation in worm stiffness is seen at a critical PEO-PBD mole fraction of X_{BD}=0.15-0.2 for blended worms of OB3/OE6. In addition to a linear increase in stiffness beyond X_{BD}=0.2 for OB3/OE6 worms, spontaneous curvature becomes locked in above the percolation point, as demonstrated in the backbone traces shown in FIG. 5. Below the percolation point, worms have a radius of curvature, which vanishes in thermal averaging (FIG. 5a). However, when the amount of cross-linked PEO-PBD is increased through the percolation mole fraction (FIGS. 5b and 5c), the increasingly solidified worm backbone fluctuates about a non-zero curvature conformation until it finally exhibits only a rigid body rotation about an easily identifiable, spontaneously curved conformation (FIGS. 5d and 5e).

The stability, loading capacity, and stealthiness of these superpolymer aggregates make them ideal assemblies for addressing questions of dynamics concerning polymeric objects, such as internal vs external viscosity effects and collective rheology of synthetic and biological systems. They also establish a foundation for focused material applications and demonstrate their utility for flow-intensive delivery applications, such as phage-mimetic drug carriers and micropore delivery, and for the creation of synthetic cytoskeletons or other structures.

Biocompatibility, encapsulation and use for drug delivery. Because of the perselectivity of the bilayer, materials may be “encapsulated” by intercalation into the hydrophobic membrane core of the worm micelle of the present invention, resulting in a “loaded” worm micelle. The term “loaded” also refers to the association of materials with the worm micelle. Numerous technologies can be developed from such micelles, owing to the numerous unique features of the bilayer and the broad availability of super-amphiphiles, such as diblock, triblock, or other multi-block copolymers.

The synthetic micelle membrane can exchange material with the “bulk,” i.e., the solution surrounding the micelles. Each component in the bulk has a partition coefficient, meaning it has a certain probability of staying in the bulk, as well as a probability of remaining in the membrane. Conditions can be predetermined so that the partition coefficient of a selected type of molecule will be much higher within the membrane of a micelle, thereby permitting the worm micelle to decrease the concentration of a molecule, such as cholesterol, in the bulk. In the alternative, worm micelles can be formed with a selected molecule, such as a hormone, incorporated within the membrane, so that by controlling the partition coefficient, the molecule will be released into the bulk when the micelle arrives at a destination having a higher partition coefficient.

By “biocompatible” is meant a substance or composition that can be introduced into an animal, particularly into a human, without significant adverse effect. For example, when a material, substance or composition of matter is brought into a contact with a viable white blood cell, if the material, substance or composition of matter is toxic, reactive or biologically incompatible, the cells will perceive the material as foreign, harmful or immunogenic, causing activation of the immune response, and resulting in immediate, visible morphological changes in the cell. A “significant” adverse effect would be one that is considered sufficiently deleterious as to preclude introducing a substance into the patient.

An enormously wide range of hydrophilic or hydrophobic materials can be associated with or encapsulated within a worm micelle, e.g., proteins and proteinaceous compositions and other carriers for drugs, therapeutics and other biomaterials, as well as marker preparations. Such encapsulate material is also referred to herein as an “encapsulant” or “active agent.” Encapsulation applications range, without limitation from, e.g., drug delivery (aqueously insoluble drugs), to optical detectors (fluorescent dyes), to the storage of oxygen, and the like.

A variety of fluorescent dyes of the type that can be incorporated within the worm micelles could include small molecular weight fluorophores, such as rhodamine. Imaging of the fluorescent core can be accomplished by standard fluorescent videomicroscopy. Permeability of the micelles to the fluorophore can be measured by manipulating the fluorescently-filled micelles, and monitoring the retention of fluorescence against a measure of time.

It is clear from the foregoing and from the Examples that follow, that worm micelles are particularly useful for the transport (either delivery to the immediately surrounding environment or removal from the immediately surrounding environment) of hormones, proteins, peptides or polypeptides, sugars or other nutrients, drugs, medicaments or therapeutics, including genetic therapeutics, steroids, vitamins, minerals, salts or electrolytes, genes, gene fragments or products of genetic engineering, dyes, adjuvants, biosealants and the like. In fact, the morphology of the worm micelles may prove particularly suited to the targeted delivery and controlled release of biocompatible compounds to a patient. They are ideal for intravital drug delivery because they are biocompatible; that is they contain no organic solvent residue and are made of nontoxic materials that are compatible with biological cells and tissues. Thus, because they can interact with plant or animal tissues without deleterious immunological effects, any drug deliverable to a patient could be incorporated into a biocompatible worm micelle for delivery.

Delivery of one or more active agents to either plants or animals, and particularly to humans, is contemplated in the present invention. Because the administration and use of a variety of drug delivery vehicles, including controlled release vehicles, is well known in the art, one of ordinary skill would know how to select and quantify the drugs or other biocompatible compositions to be delivered to a patient, as well as methods for administering the loaded worm micelles of the present invention to a patient and monitoring the release of the one or more active agents and finally the removal of the fragmented worm micelles from the patient's system.

Adjustments of molecular weight, composition and polymerization of the micelle can be readily adapted to the size and viscosity of the selected drug by one of ordinary skill in the art using standard techniques, and the release of an encapsulated active agent can be controlled by the length of the worm. Once the encapsulated active agent has been released and the worm has been fragmented, it is then quickly removed from the patient's circulation.

In bioremediation, the worm micelles could effectively transport waste products, heavy metals and the like. In electronics, optics or photography, the worm micelles could transport chemicals or dyes. Moreover, these stable micelles may find unlimited mechanical applications including insulation, electronics and engineering. Additional encapsulation applications that involve incorporation of hydrophobic molecules in the bilayer core include, e.g., alkyd paints and biocides (e.g., fungicides or pesticides), obviating the need for organic solvents that may be toxic or flammable. Worm micelles also provide a controlled microenvironment for catalysis or for the segregation of non-compatible materials both in vivo and in vitro.

The present invention is further described in the following examples in which experiments were conducted to characterize the worm micelles. These examples are provided for purposes of illustration to those skilled in the art, and are not intended to be limiting unless otherwise specified. Moreover, these examples are not to be construed as limiting the scope of the appended claims. Thus, the invention should in no way be construed as being limited to the following examples, but rather, should be construed to encompass any and all variations that become evident as a result of the teaching provided herein.

### EXAMPLES

### Example 1

### Visualization of Stable, Stiffness-Tunable, Flow-Conforming Worm Micelles

To visualize and characterize the formed and highly stable worm micelle superpolymers in aqueous solution, two worm-forming diblocks (structural details in Table 1) were examined—one with an inert hydrophobic block of PEE (polyethylethylene), designated OE6, and another with cross-linkable PBD (polybutadiene), designated OB3. The selected block copolymer amphiphiles have molecular weights (MW) that are 5-10 times that of typical lipids (Won et al., supra, 1999; Won et al., supra, 2001), which highlights the stability imparted by the blended copolymers.

To demonstrate control over worm shape and flexibility, the assemblies were labeled by adding fluorescent dye, which incorporates into the hydrophobic cores of the worms in a matter of seconds. Fluorescence video microscopy has, of course, been applied to study the dynamics of DNA (Maier et al., Phys. Rev. Lett. 82:1911 (1999), microtubules (Gittes et al., J. Cell Biol. 120:923 (1993), and F-actin (Ott et al., Phys. Rev. E 48:1642 (1993), but the selected biopolymers are complex polyelectrolytes. Solution ionic strength, multivalent ions (e.g., Ca), metabolic substrates (e.g., ATP), contaminating cross-linker proteins, and oxidation (e.g., disulfide cross-links) (Tang et al., J. Biol. Chem. 271:8556 (1996)) are among the most compounding of factors, and all can profoundly influence polymer dynamics, aggregation states and collective rheology.

The copolymers OB3 and OB6 (see Table 1) were synthesized using anionic polymerization techniques as described by Hillmyer et al., supra, 1996. The number of monomer units in each block was determined from ¹H NMR. The molecular weights of the copolymers were determined using gel permeation chromatography with polystyrene standards. Thus, the selected PEO-PEE and PEO-PBD worm systems have a simpler composition of strongly segregating, nonionic copolymers with just two monomer types (three when blended) plus dye, as opposed to 19 amino acids in proteins or four base pairs in a complex DNA sequence, plus counter-ions, dyes, etc.

Worm micelles were formed in water at 55-60° C. using film hydration techniques (see, Menger et al., Acc. Chem. Res. 31:798 (1998)). Thus formed by stirred hydration of dried films of the nonionic block copolymer amphiphiles, the worm micelles self-assembled. The disclosed methods of preparation of the micelles are particularly preferred because the vesicles are prepared without the use of co-solvent. Any organic solvent used during the disclosed synthesis or film fabrication method has been completely removed before the actual vesicle formation. Therefore, the worm micelles of the present invention are free of organic solvents, distinguishing the worm micelles from the prior art and making them uniquely suited for bio-applications. By blending and polymerizing inert and cross-linkable copolymers, the resulting micelles were up to tens of microns long with persistence lengths that continuously spanned more than 2 orders of magnitude from submicron to submillimeter. The worms were then labeled with a hydrophobic fluorescent dye (PKH26; Sigma, St. Louis, Mo.) in a molar ratio of 1:3 (dye:copolymer). The dye was added directly to the aqueous solution of worms.

The backbone contours of the worms were visualized with a Nikon TE-300 inverted fluorescence microscope (TRITC; Chroma Technology Corp., Rockingham, Vt.). The worms were confined between a coverslip and a slide, which were spaced ˜1 μm apart using polystyrene beads as spacers. Video-rate images of the worms were collected with an ICCD camera (Princeton Instruments, Roper Scientific, Inc., Trenton, N.J.).

Stability and Flexibility. Because of the kinetic barrier to further aggregation and the strong association of the large hydrophobic blocks, worms tend to be stable assemblies as indicated by the contour length distribution (FIG. 1, inset) which persists for at least a month. Fits of such a distribution yield a minimum self-association energy α≧26k_{B}T, which is indeed far higher than the α10Ok_{B}T, which is typical of small amphiphile assemblies (see, Israelachvili, In Intermolecular & Surface Forces, Academic Press: London, 1992, p 353).

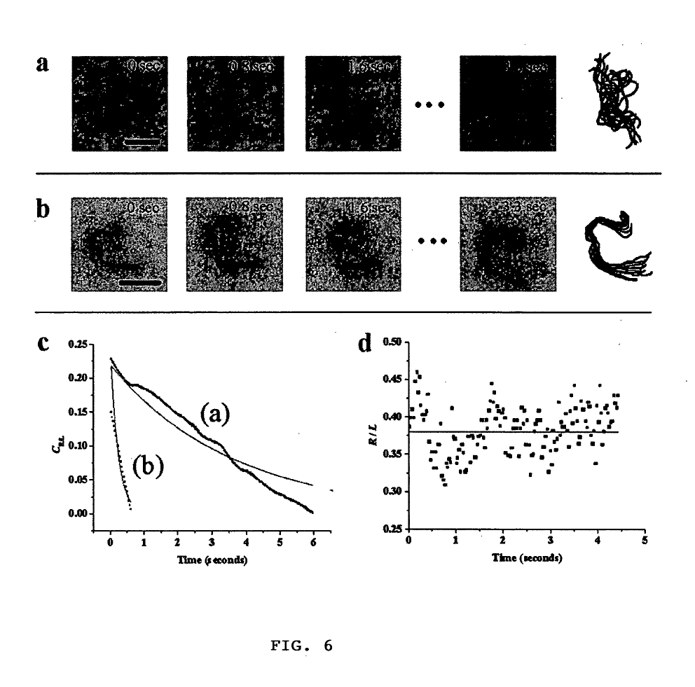

For pristine worms of OB3 or OE6, time sequence snap-shots and overlays of skeletonized contours demonstrated the considerable flexibility of this fluid system (FIG. 6a). An autocorrelation time for relative end-to-end length (R/L) of τ_{R/L}=2.6±0.6 seconds, allows for accurate visualization as well as adequate statistical measures, when distances are measured from fluorescence video images. A self-avoiding chain fluctuating in d*-dimensions was expected to increase in size as <R>=a^{1-θ}L^{θ}, where α is the blob size in a bead-on-string model, and the well-known scaling exponent θ=3/(2+d*). Since the worms here are confined to a ˜1 μm gap defining a pseudo-two-dimensional geometry, it was expected that θ≈¾, whereas it was found that θ=0.84±0.06. This is in close agreement with θ=0.79±0.04 found previously for DNA adsorbed to a cationic lipid membrane (Maier et al., supra, 1999).

The persistence length of the noncross-linked worms is l_{P}=500±200 nm (estimated from the relation <R²>=2l_{P}−1+exp(−L/l_{P})), where I_{P} is divided by two since the worms are confined to a pseudo-2D gap (Grosberget al., Statistical Physics of Macromolecules, AIP Press, New York, 1994, p 10; Wilhelm et al, Phys. Rev. Lett. 77:2581 (1996)). This proved to be the same as l_{P} (=570 nm) obtained from small-angle neutron scattering data fit to the Kratky-Porod model (Flory, supra, 1969), implying that dye labeling is minimally perturbing. The persistence length of a self-assembled worm proves useful to understanding the molecular basis for its rigidity, noting that l_{P}=κ/k_{B}T, where κ is the bending rigidity. A D-dimensional object (e.g., worm or membrane) whose rigidity is dominated by interfacial tension has κ=θγd^{4-D}, where θ is an interfacial coupling constant, γ is the interfacial tension, and d is the diameter of the hydrophobic core. For a worm, D equals 1, and for a membrane, D equals 2. In a separate study (not shown) of a MW series of worm-forming copolymers, κ˜d^{2.8}.

Recently described nanofibers (Liu et al., Macromolecules 36:2049 (2003)) composed of polystyrene-polyisoprene diblock copolymers in tetrahydrofuran (THF) have been reported to have l_{P} s ranging from ˜150 to ˜620 nm based on bulk light scattering and viscometry. An estimated core diameter of d≈12 nm from thin film characterization of the nanofibers would indeed suggest a slightly lower l_{P} than that found here where d≈14 nm. In addition, THF (as opposed to water) in the nanofiber system is likely to give a smaller γ, which also tends to reduce l_{P} of the cited nanofibers.

Using γ=25 pN/nm as a typical interfacial tension for the present block copolymer assemblies (Discher et al., J. Phys. Chem. B 106:2848-2854 (2002b)) and d=14 nm (Won et al., supra, 1999), when κ=k_{B}Tl_{P}, then θ=0.05. This θ is slightly greater than that found for either lipid bilayers (˜0.03) (Goetz et al., Phys. Rev. Lett. 82:221 (1999)) or polymer vesicle membranes (˜0.02)(Discher et al., supra, 1999), suggesting that the worm core is effectively well coupled or inter-digitated, even in the absence of copolymer cross-linking.

Cross-Linking in Core-Percolated Rigidity. While an non-cross-linked worm of either OB3 or OE6 explores much of the configuration space available to it in a few seconds (FIG. 6a), a fully cross-linked worm of OB3 barely flexes, and instead exhibits rigid-body rotation (FIG. 6b). Free radical polymerization, as used in the present assemblies, uses a water-soluble redox combination (see, e.g., Odian, Principles of Polymerization, 3rd ed., Wiley: New York, 1991, pp 216-217) of potassium persulfate (K₂S₂₈) and sodium metabisulfate (Na₂S₂O₅)-ferrous sulfate (FeSO₄.7H₂O) under well-stirred conditions. A very similar PEO-PBD system (EO37-BD28), which generates worms and vesicles coexisting in water, has instead been cross-linked by γ-irradiation with a ⁶⁰Co source, and has also been proven stable in THF (Maskos et al., Macromol. Rapid Commun. 22:271 (2001)). Complete cross-linking of the present system has been verified elsewhere by testing the chloroform extractability of copolymer (Won et al., supra, 1999).

Kinks and highly contorted shapes are locked-in upon cross-linking (FIG. 6b). Dynamic bending modes persist nonetheless and are significantly faster after cross-linking: τ_{R/L}=0.2±0.1 seconds (FIG. 6c). Estimation of l_{P} for such rodlike objects where l_{P}>>L has been the subject of prior study (Wilhelm et al., supra, 1996; Gittes et al., supra, 1993, wherein long microtubules (L˜30 μm) with persistence lengths known to be millimeters were analyzed by a Fourier analysis of the thermally excited bending undulations). Each normal mode has a thermal energy of 1/2k_{B}T, and the sum of modes is cumulatively resisted by the strain energy to bending the rod, which is scaled by the bending rigidity κ. With the locked-in shapes of cross-linked OB3, the spontaneously curved average shape was subtracted, and Fourier analysis was performed on the first three modes since they deviate from the average shape.

Surprisingly, perhaps, the system appeared ergodic (FIG. 6_{d}), with each mode adequately accessed. Note also that τ_{R/L} reported above was small compared to the minimal analysis time of ˜3 seconds. The normal-mode analysis (Gittes et al., supra, 1993) yielded 3D κ=0.46 pN μm², and l_{P}=115±30 μm. This agrees with small-angle neutron scattering results on these cross-linked worms, despite the potential limitations of such scattering and the need to assume a models in this case a solid beam (Won et al., J. Phys. Chem. B 104:7134 (2000)).

Indeed, for a solid beam κ˜d⁴, rather than d³ as above for pristine worms. The Young's modulus, E, of a solid worm with a cross-linked hydrophobic core can also be obtained from l_{P}=EI/k_{B}T, where I=π(d/2)⁴/4 is the cross-sectional moment of inertia (Gittes et al., supra, 1993) and d=12.4 nm is the diameter of a cross-linked worm (Won et al., supra, 1999). This yields E=400 MPa (mega-Pascal), which far exceeds moduli obtained with macroscopic samples of cross-linked polybutadiene (<10 MPa) and indicates that confined cross-linking of the present invention is extraordinarily efficient.

Blending of copolymers before hydration followed by cross-linking leads to worms containing both inert PEO-PEE (OE6) and cross-linked PEO-PBD (OB3). This gave l_{P}'s that range continuously from ˜0.5 to 100 μm, which is equivalent in range to the bending rigidities of biopolymers as diverse as neurofilaments (Janmey et al., J. Cell Biol. 113:155 (1991)), F-actin, and (almost) microtubules (Gittes et al., supra, 1993). Fluctuation dynamics of the partially cross-linked systems (as measured by the relaxation time, τ_{R/L}) also show a strong dependence on the cross-linking mole fraction, X, of OB3 in OE6. For X below the percolation point X_{C} (≈0.15 mole fraction OB3 in OE6), τ_{R/L} is relatively constant, whereas for X greater than X_{C}, τ_{R/L} decays exponentially (FIG. 6c). This decay above X_{C} reflects the increasing stiffness of an overdamped system, and the lack of a strong peak in τ_{R/L} near X_{C} indicates that the viscosity of the core of a worm is not a significant factor in worm dynamics. Below X_{C}, relaxation can thus be attributed to hydrodynamics of the embedding aqueous media.

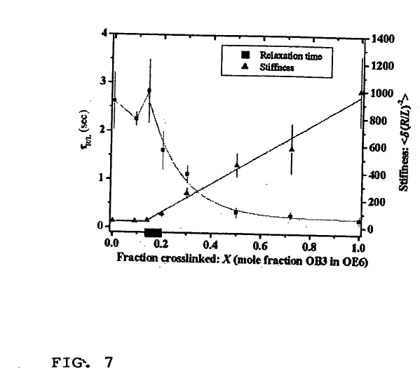

When worm micelle dynamics and stiffness were measured as a function of cross-linking, the effective stiffness of partially cross-linked worms formed by copolymer blending was revealed in the end-to-end length-normalized fluctuations of a worm, i.e., <δ(R/L)⁻²) (FIG. 7). By this metric, covalent cross-links began to percolate along the hydrophobic butadiene core at the same X_{C} (≈0.15). As shown in FIG. 7, worms with cross-linked OB3 in OE6 (OB3 mole fraction, X) below the percolation point X_{C}(X<X_{C}) have very similar relaxation times, τ_{R/L}, for normalized end-to-end length, R/L, and the worm was clearly a fluid. Worms above X_{C}(X>X_{C}) were solid with a linear increase in effective stiffness, having a decreasing τ_{R/L}˜τ₀exp[−ξ(X−X_{C}], where τ₀ is the average relaxation time below percolation and ξ≈7 is the damping coefficient representing the increase in internal viscosity. The effective stiffness of the partially cross-linked worms is given by the fluctuations in R/L, <δ(R/L)⁻²>=(<R/L)⁻²<−<(R/L)⁻²>. For X<X_{C}, worms have very similar stiffness, whereas for X>X_{C}, worm stiffness increases linearly with a slope of 11 (FIG. 7).

Phase separation of the two copolymers seems unlikely since the error bars are not large for <δ(R/L)⁻²>, and no discontinuities in stiffness along single blended worms were observed (Discher et al., supra, 2002b). The same X_{C} and linear increase in elasticity for X>X_{C} have been observed in fluid-to-solid transitions of cross-linked polymer vesicle membranes (Discher et al., supra, 2002b) that are assembled from similar blends of copolymers with suitably reduced w_{EO} (i.e., w_{EO}˜25-45%).

Flow Stretching. Theories for stretching Gaussian chains under moderate flows in various geometries (Brochard-Wyart, Europhys. Lett. 23:105 (1993)) have already been tested by experiments on single DNA molecules (Perkins et al., Science 268:83 (1995)). While holding one end fixed, large extensions in the direction of flow (>30% of L) have shown that the fractional extension x/L of DNA scales with vL^{ω}(ω=0.54±0.05), where v is the mean flow velocity. This scaling is shared, in theory, by all semiflexible polymer systems (hence universal) since a modified worm-like chain model accounts for the experimental data at least for chains up to L≈40 μm (Marko et al., Macromolecules 28:8759 (1997); Larson et al., Phys. Rev. E. 55:1794 (1997)). Universality was experimentally confirmed, since the directional elongation of pristine worms scales as vL^{0.52}±0.01 (see, Dalhaimer et al., Macromolecules 36:6873-6877 (2003)). Mean flow velocity, v, was estimated from smaller micelles flowing past. Snapshots and backbone traces of noncross-linked worms extended under various flow velocities also appear qualitatively consistent with theoretical “trumpet” shapes (Brochard-Wyart, 1993), which become increasingly narrowed and stretched out under high flow (although Re<1)(Re refers to Reynolds number, see Purcell, Amer. J. Phys. 45(1):3-11 (1977); Dalhaimer et al., Controlled Release Physique 4:251-258 (2003)).

Importantly, the worms did not fragment under flow-imposed tensions that were estimated to reach ˜1 μN/m. Tension on worms is calculated from a plane Poiseuille flow model with a velocity profile vₓ(y)=3v[1−(y/H)²], wherein v is the average flow velocity, y is the distance between coverslips, and H is the gap height. The tension is the shear stress (μ∂vₓ/∂y; μis viscosity) integrated over the contour length of each worm. While not an issue for covalent polymers, such as DNA, integrity under stress is in line with the stable distributions of worm persistence lengths shown in FIG. 1c, indicating that this is more of a stable aggregate than a living polymer.

Consistent with cross-link-enhanced stability but contrasting with the trumpet-shaped envelopes, the backbones of totally cross-linked worms are largely unaffected by flow (Dalhaimer et al., supra, 2003). Spontaneous curvature of these cross-linked micelles is truly locked in while the worm thermally fluctuates with or without externally imposed forces. If minimization of kinks is desired for a particular application, cross-linking can be done while either stretching worms under shear, as shown here, or lyotropically straightening for a particular application the worms in dense solutions of mesogens of the same or dissimilar type (e.g., rodlike phages or viruses). Pristine worms, and polymeric objects in general, also tend to stretch out when flowing through a confining structure, such as a pore or a microcapillary.

Aspiration of a pristine worm into a 4 μm diameter glass capillary tube showed initial diffusion in bulk within a chamber filled with blood cells (v≈35 μm/s), and in a period of less than 3 seconds the worm transitioned from entering the capillary tube from the bulk to becoming fully extending, convectively aligning, and traveling down the tube (Dalhaimer et al., supra, 2003). The worm extension behavior in the confined capillary flow is the inverse of that shown in FIG. 1b, and shows that pristine worms are equally as adept at stealthily traveling in complex fluid flows as they are at diffusively expanding into a stagnant zone or cavity. Thus, the worms are stable among cells and in blood plasma composed of thousands of proteins, fatty acids, and other surface active agents. This inertness undoubtedly stems from the stealthiness imparted by the PEO, which is absolutely essential to worm formation. This is in marked contrast to PEG lipid, which is a mere additive in the so-called “stealthy” liposomes.

Therefore, worm micelles assembled from PEO-based nonionic copolymers have proven to be extremely stable in aqueous media, and the dynamics of the worms can be directly visualized with fluorescence microscopy techniques. In flow, pristine worms orient and stretch with DNA-like scaling. The fluorescence microscopy images taken over a time sequence in FIG. 1b, show a labeled, highly stable worm extending in flow among obstacles (I)s (in this case blood cells). The worm then expanded (II) and sequestered in a stagnant zone (III). The presence of a brushy PEO corona also tends to sterically stabilize these assemblies and minimize interactions with other worms and surfaces. While illustrative of the stability, flexibility, and convective response of worms, such results are also demonstrate the utility of such structures for micro- or nano-applications when flow is highly directing in micellar drug delivery and pore chemistry and motivates a deeper understanding of the dynamics associated with such worms, which are an intermediate microphase, as compared with vesicles and spherical micelles, both of which have been widely studied.

### Example 2

### Targeting and Delivery of Hydrophobic Drugs

In light of the stability, flexibility and convective responsiveness of the worm micelles shown in Example 1, the ability of the worms to target and deliver hydrophobic drugs to a host cell was studied using principles analogous to those used in viral delivery of DNA (Gref et al., supra, 1994). In vitro targeting and in vivo circulation assays were utilized to evaluate the functional capabilities (e.g., the ability to bind to cells and transfer encapsulated contents) of the worm micelles using biotin and a small ligand that binds to a receptor that is generally upregulated on tumor cells. Biotinylated macromolecules have been previously shown to undergo receptor-mediated endocytosis both in animal and plant cells (Photos et al., supra, 2003; Pasqualini et al., supra, 1996).

Hydroxyl ends of PEG-PEE block copolymers were made amine-reactive using p-nitrophenylcarbonyl (NPCF) modification chemistry (Lasic et al., supra, 1996). Briefly, 0.2 g of PEG-PEE copolymer was dissolved in 1 ml of dimethylene chloride on ice. Added to the mixture were 8 μl of pyridine and 30 mg of 4-NPCF, and the solution was stirred overnight on ice. Next, 15 ml of an ice cold mixture of ethyl alcohol and HCl at a ratio of 40:1 was added, making the solution turbid. The solid was precipitated from the solution at −20° C. for 15 minutes. The resulting mixture was spun down at 6000 rpm for 8 minutes at 4° C., after which the pellet was broken up and 15 ml of an ice cold mixture of ethyl alcohol and HCl at a ratio of 160:1 was added. The centrifugation was repeated and 15 ml of ice cold, pure ethyl alcohol was added to the solution. The resulting PEE-PEG-NPCF copolymer (biotinylated PEG₄₀-PEE₃₇ copolymer (m˜37, n˜40, p˜9)) was dried under vacuum.

For the biotinylation reaction, the PEE-PEG-NPCF copolymer was dissolved in 10 mM citrate at pH 4. Biotin-PEG-amine (MW 720; Sigma-Aldrich, St. Louis, Mo.) was added in molar excess to the copolymer and the pH was raised to 8.5 with NaOH, at which point the solution turned yellow. The solution was then incubated at room temperature for 2 hours. The biotinylated copolymer was separated from the free Biotin-PEG-amine by dialysis using a 2000 Da cutoff membrane. Diblock copolymers with 25% biotinylated copolymer blended as a solution of CHCl₃ were assembled into worm micelles at 60° C. via film rehydration with PBS, as in Example 1.

Worm micelles were visualized with a hydrophobic PKH26 dye (Sigma-Aldrich), as follows. Fluorescene-strepavidin (Molecular Probes, Eugene, Oreg.) was mixed in BSA at 0.1% (mol) and adsorbed to a glass cover slip. Excess protein was washed off with phosphate buffered saline (PBS). A 50 μl solution of PKH26-labeled worm micelles was then added to the surface and washed off after 15 minutes. Rhodamine fluorescent images were then taken of the surface and the number of adsorbed worm micelles was counted. Schematically the biotinylated worm micelles appear as shown in FIG. 1a, wherein the spheres in the core of the micelle represent PKH26 fluorescent dye molecules, but where covalently attached biotin molecules bind to the hydrated ends of a fraction of the copolymer chains (not shown in FIG. 1a).

Stability. Length distributions of biotinylated worm micelles (25% biotin copolymer) formed by simple hydration of dried films proved to be stable for at least several weeks, which as a point of reference is a much longer period than the time scale for in vivo circulation of related copolymer vesicles. Thus, stability was not considered to be a problem for in vivo applications.

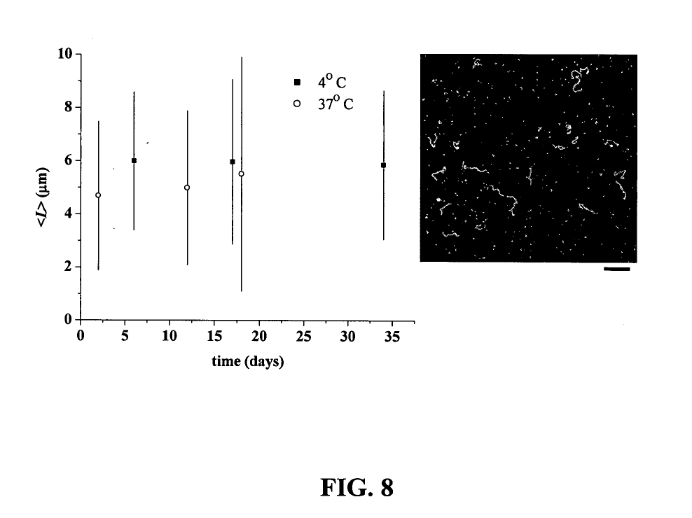

FIG. 8 shows a plot of average contour length (L) as a function of time at 4° C. and 37° C., showing them to be stable for at least a month. Because of optical resolution limitations with microscopy, contour lengths were measured only for worm micelles with L>1 μm. The inset of FIG. 8 shows biotinylated worm micelles bound to an avidin-coated surface after micelle storage for one week.

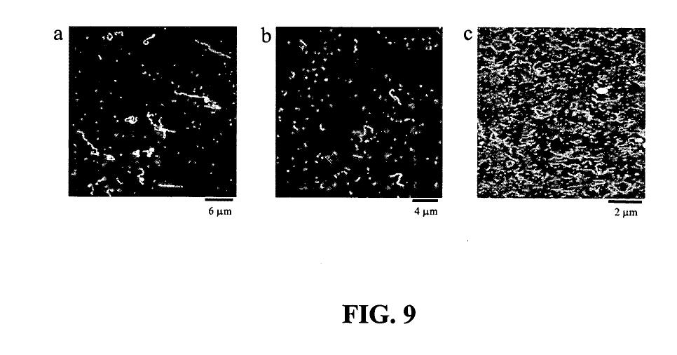

Since micron-long worm micelles might be anticipated to disrupt flow in the vasculature, it seemed advantageous to anticipate a need for control over the contour lengths of the worm micelles. FIG. 9 shows a series of fluorescence and atomic force microscopy snapshots of biotinylated (25%) and pristine worm micelles adsorbed to surfaces after short sonication times. An additional method involved repeated extrusion of the worm micelles through a 400 nm filter at pressures of ˜200 psi. This latter method is perhaps preferred for controlling the contour lengths of the worms since it also removes micron-size particles that could obstruct the vasculature.

Interactions with avidin-coated surfaces. In contrast with pristine worm micelles, biotinylated worm micelles showed strong binding (at least two orders of magnitude more frequently) to 0.1% (mol) avidin/BSA-coated surfaces (not shown). The molar ratio of adsorbed avidin to BSA was intended to mimic the density of receptors on a cell surface with a spacing of ˜50-100 nm. Biotinylated worm micelles zipped-up on the avidin-coated surfaces. This was in contrast to pristine worm micelles, which either did not stick at all, or attached only at or near one end. Neither worm sample showed any binding to BSA-coated surfaces(alone).

Interactions with cells. Since biotin is a vitamin that is internalized by most cells (Vesely et al, Biochem. Biophys. Res. Comm. 143:913 (1987); Horn et al., Plant Physiol. 93:1492 (1990)), the cell-targeting capability of biotinylated worm micelles was tested by addition to cultures of smooth muscle cells. Smooth muscle cells (SMCs) of the A7r5 lineage (rat aorta-derived) were maintained in polystyrene flasks between passages 2 and 15, and cultured in growth media (Dulbeco's Modified Eagle (DMEM), supplemented with 10% of fetal bovine serum (FBS), and antibiotics (penicillin and streptomycin) (all from Invitrogen, Carlsbad, Calif.)). Cells were passed every 3 days at ˜80% confluency, and plated on 35 mm dishes with a circular coverslip (MatTek Corp., Ashland, Mass.) to enable in vitro microscopy.

Prior to addition of drug, soluble reagent, or worm micelle, cells were plated at ˜1×10⁴ cells/dish and grown for approximately 24 hours. Worm micelles were incubated with the SMCs for 4 hours, and then analyzed by fluorescence microscopy for binding and internalization.

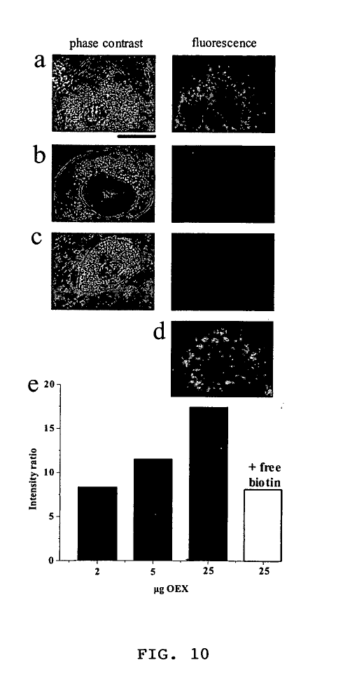

After confirming that fluorescein-biotin bound to these SMCs (data not shown), biotinylated worm micelles at two different concentrations were then incubated with the cells for 4 hours. FIG. 10 shows phase contrast and fluorescence images comparing smooth muscle cells that were incubated with biotinylated worm micelles, with those incubated with pristine worm micelles, or with those control cells not exposed to any worm micelles. Cells incubated with biotinylated worm micelles (labeled with a PKH26 hydrophobic tag) appeared 10-fold brighter on average (FIG. 10d) than cells incubated with pristine worm micelles (FIG. 10b) or no-addition controls (FIG. 10c). A bar graph is presented in FIG. 10e to show comparative intensity measurements on at least 10 cells, showing that cells incubated with biotinylated worms appear on average 10-fold brighter than cells incubated with equal concentration of pristine worms. Fluorescein (green) images showed little difference in intensity between control cells and the various cells incubated with worm micelles, demonstrating that broad-spectrum auto-fluorescence was not a problem with these cells.

The nucleus was generally clear in these cells, and the biotin-targeted fluorescence was perinuclear, providing clear evidence of internalization, rather than mere surface binding. Since no large worm micelles were seen in any of the cell cultures with biotin-PEG-PEE, the micron-long worm micelles were evidently being imported, and perhaps fragmented as they entered these cells. To confirm this finding and eliminate the possibility of an error caused by the labeling system, the experiment was repeated using biotinylated worm micelles prepared using a rhodamine tag that was covalently bound to the PEG hydroxyl. Nevertheless, the cell images showed no qualitative difference in fluorescence distribution when the rhodamine-labeled worm micelles were added to the cell cultures. Thus, there can be no question that the worm micelles were actually internalized through biotin-receptor endocytosis.

Delivery applications require specificity of carrier to target. The strong affinity of biotin to avidin made biotinylated worm micelles a reliable indicator of covalent modification. Therefore, with living cells, the biotinylated worm micelles have been shown to both bind to cell receptors, and transfer hydrophobic cargo (fluorescent dye) out of their cores across the plasma membrane to a generic cell line. By comparison, in both cases the pristine worm micelles that were not functionalized, showed little interaction. Accordingly, other cell-specific ligands can also be attached to the worm micelles to target specific delivery of encapsulated molecules (e.g., drugs), including micron-long filamentous phages that infiltrate tumors in vivo and specifically bind via displayed peptides.

### Example 3

### Synthetic Vehicles for Drug Delivery

Many factors are involved in the circulation time of a foreign object through the vasculature, however, the role of vehicle shape and stability in clearance has remained indefinite. To answer that question, the following experiments were designed to confirm that the micron length, flexible, worm micelles of the present invention, have a significantly longer circulation time than previously utilized delivery vehicles, and to demonstrate their ability to load and transport a hydrophobic encapsulated material (i.e., drug) to a specific cell receptor.

In vivo circulation. To take advantage of their unique flow properties, flexible, micron-length cylindrical worm micelles were prepared as described above in Example 1 from two ˜5000 MW worm-forming PEG-based copolymers—OEX and OB3 (see, Table 1). Blends of these two copolymers formed cylindrical worm micelles with diameters of ˜10 nm and average contour lengths, L, of ˜10 um. The persistence lengths of worm micelles formed from these copolymers was calculated in Example 1, to be ˜500 nm, which is important for conforming to streamlines in flow through capillaries. Thus, the worm micelles have the loading capacity of a micron-length object, with the stealthiness of a ˜10 nm width object.

OEX was modified via p-nitrophenylcarbonyl chemistry (Torchilin et al. Biochim. Biophys. Acta 1511:397 (2001)), wherein the final copolymer (OEX_{hTfR}) contained a 12 amino acid peptide (THRPPMWSPVWP), which has previously been shown to bind to human transferrin receptor (hTfR) (Lee et al., Eur. J. Biochem. 268:2004 (2001)). hTfR is generally up-regulated on proliferating tumor cells (Miyamoto et al., Int. J. Oral Maxillofac. Surg. 23:430 (1994); Keer et al., J. Urol. 143:381 (1990)). OEX_{hTfR} was blended into the worm micelles at 1% total number of OEX copolymers per worm, which was sufficient to functionalize an aggregate.

Worm micelles used for in vivo assays were a 10% molar blend of OB3 in OEX, wherein OB3 was used to ensure that no vesicles were part of the aggregate population. Sprague-Dawley (SD) male rats were injected with ˜0.5 ml of 2.5 mg worm micelles. Samples were first run through a 400 nm membrane, both to eliminate particles that could obstruct capillaries and also to maintain a maximum worm contour length of ˜5 um. Orbital bleeds were taken at various times during the study to determine the mass (proportional to intensity) of copolymer aggregate in circulation.

Worm micelles were incubated with the cells, typically for 4 hours, after which, the media and unbound worm micelles were removed with 10 washes using PBS. For in vivo assays, worm micelles were visualized with PKH26, as in Example 1. The worms were loaded with a fluorescein-conjugated taxol in a molar ration of marker to copolymer of 1:14. Images were recorded using an Olympus IX71 inverted microscope with a CCD camera (Cascade, Roper Scientific, Inc., Tucson, Ariz.).

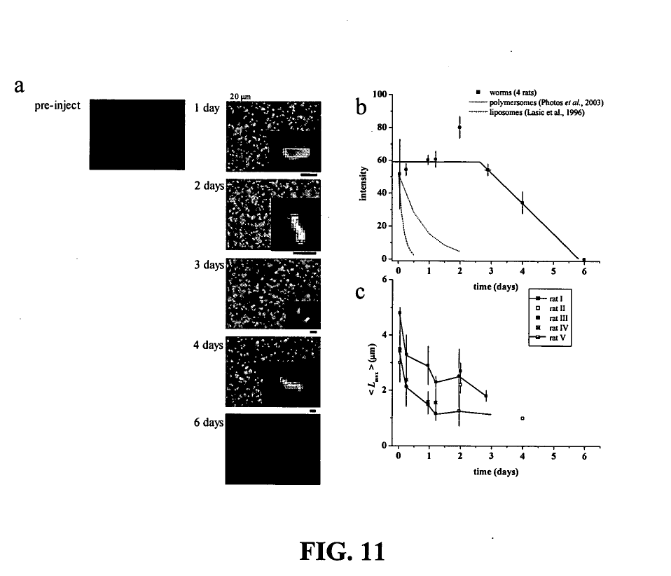

Upon close examination in the longer time samples, it was obvious that the majority of the surviving aggregates were worm micelles. FIG. 1 la shows snapshots of fluorescently labeled worm micelles withdrawn from rat circulation over several days. FIG. 11b shows circulating mass (fluorescence intensity) of worm micelles as a function of time in male SD rats. In FIG. 11c, the contour lengths were averaged of the five longest worm micelles seen in the sample. Of course, the average contour length of the worm micelles in circulation would be much smaller than the numbers reported in FIG. 11c, but it was not possible to measure sub-micron lengths in the presence of plasma proteins. As shown, worm mass stayed relatively constant over a period of three days, but once the worm micelles were broken down to the sub-micron size of vesicles, they followed the clearance behavior of PEGylated spherical objects, decaying at approximately the same rate as 100 nm polymersomes (FIG. 11c).

Copolymer (OB18) used to form the polymer vesicles has a PEG volume fraction that also allowed for the formation of worm micelles. However, physically the OB 18 worm micelles have a much larger diameter (d˜27 nm) and persistence length (l_{P}˜6 um) than the OEX worm micelles (d ˜11 nm; l_{P}˜0.5 um). The OB18 worm micelles were compared at 1 hour and again at 48 hours after injection into a male SD rat. Significantly, the average contour length of the five longest worm micelles remained constant over this time period (not shown) with <Lₘₐₓ>₁ₕᵣ=5.7±2 and <Lₘₐₓ>₄₈ₕᵣ=5.4±1. No decay was seen in any of the 5 longest average contour length OB18 worm micelles. This stability in contour length contrasted with the OEX worm micelles, which showed a strong decay in contour length over the same time period (FIG. 11c). Thus, the larger diameter OB18 worm micelles were more stable and less susceptible to fragmentation.

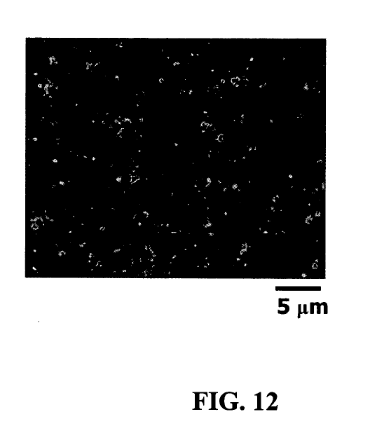

In vitro assays. Drug delivery with liposomes entails loading either hydrophilic drugs or nucleic acids into the interior of the assembly (Wagner et al., Proc. Nat'l Acad. Sci. USA 89:7934 (1992)), or hydrophobic drugs into the lipid bilayer (Wenk et al., J. Pharm. Sci. 85:228 (1996)). The worm micelles in this study were loaded with the drug taxol, one of a variety of hydrophobic drugs available for killing tumor cells. Taxol binds to, and toxically stabilizes, microtubules at 1-10 nM (Gloushankova et al., Proc. Nat'l. Acad. Sci. USA 91:8597 (1994)). In the worm micelles, one molecule of taxol partitions into the hydrophobic core per ten copolymers as confirmed by HPLC (data not shown). Worm micelles were visualized for in vitro assays using the hydrophobic marker, Oregon Green paclitaxel (taxol) (Molecular Probes, Eugene, Oreg.), and fluorescein conjugated taxol was used to confirm loading of the drug into the worm micelles (FIG. 12) and to study the transfer of the drug into cells, and ultimately to the cytoskeleton.

Worm micelles loaded with Oregon green paclitaxel, and functionalized with a human transferrin receptor binding peptide, THRPPMWSPVWP, were used with 3 cell lines in vitro for targeting transferrin. The model lines were: chicken embryo fibroblasts (CEF) lacking human hTfR, chicken embryo fibroblasts transfected with hTfR (CEF-hTfR), and A549 human lung carcinoma cells, which have a naturally high expression of transferrin due to tumor proliferation. CEF and CEF-hTfR cell lines were cultured in media containing DMEM with sodium pyruvate, 2% chicken serum, 2% fetal bovine serum (FBS), 2% tryptose pyruvate, 1% L-glutamine, 1% 50 mM HEPES buffer, and antibiotics. CEF cells were passaged every 2 days, whereas CEF-hTfR cells were passaged every 4 days, or when either reached ˜80% confluency.

Cells were cultured on polystyrene flasks between passages 2 and 15, and were plated at ˜1×10⁴ cells/dish on glass-bottom 35 mm wells (Mattek Corporation) to enable convenient in vitro microscopy for experiments. Cells were plated and incubated for approximately 24 hours prior to the addition of a drug, soluble reagent, or worm. As above, all other cell culture products were from Invitrogen.

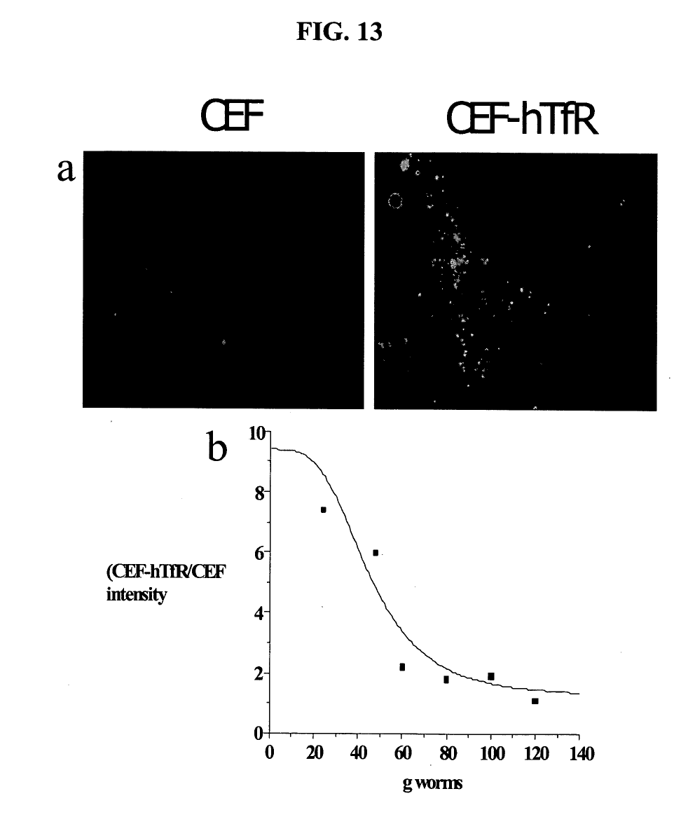

The labeled worm micelles, functionalized with the human transferrin receptor binding peptide, THRPPMWSPVWP, were incubated with CEF and CEF-hTfR cell lines for 4 hours (FIG. 13a). At high concentrations of worm micelles, non-specific binding reduced the difference in intensity between cells. FIG. 13b shows the saturation limit of hTfR at ˜50 μg of copolymer, above which, worm micelles bound to CEF cells non-specifically.

To rationally model and fit this data, it was assumed that the worm micelles bind saturably with a specific dissociation constant Kₛ and a non-specific dissociation constant Kₙ and a common cooperativity exponent μ. Accounting for different levels of total specific and non-specific binding with a constant A, the relative amount of binding at a given worm micelle can be shown to have the form

$${Θ}_{Rel} = 1 + A ( \frac{{K}_{n}^{μ} + {C}^{μ}}{{K}_{s}^{μ} + {C}^{μ}} ) .$$

One other common assumption was the strong cooperativity assumption with μ>1 in an all-or-none type of process with respect to suitable clusters of cell surface receptor sites. A four parameter fit of the data in FIG. 13b with (A, Kₙ, Kₛ, μ) was done to minimize R². The reasonable fit shown gives A=0.21, Kₙ=126 μg, Kₛ=45 μg, μ=3.54. The fact that μ>1 is consistent with cooperative zipping-up on the cell surface, and Kₛ<Kₙ is consistent with reasonable specificity. Since A<1, however, the number of hTfR sites on these cells is small relative to the less-specific sites.

Finally, the human lung cancer cell-line A549, showed significant binding with hTfR peptide-functionalized worm micelles versus worm micelles lacking the peptide (data not shown). Thus, worm micelles can carry hydrophobic cargo to a specific delivery site through covalent modification of the PEG brush with a specific receptor-binding ligand. In vitro assays demonstrated that worm micelles can be loaded with a cytotoxic drug (paclitaxel), and deliver the drug via a specific targeting peptide to tumor cells expressing selected human receptors by ligand-receptor binding. Consequently, the cylindrical geometry of the worm micelles provides an intriguing alternative to spherical carriers since worm micelles remain in the vasculature of a mammal (up to almost a week after injection), which is significantly longer than spheres. While the fluidity of the worm micelles makes them susceptible to fragmentation in circulation, the more stable worm micelles tend to maintain their contour length over time, thereby predicting targeted in vivo exploitation of these assemblies.

Thus, the giant and stable worm micelles formed from PEG-based diblock copolymer amphiphiles of the present invention compare favorably to structural biopolymers in terms of bending rigidity and contour length. Through chemical modification of the exterior of the worm micelles, they are integrated into a cellular environment in the context of drug delivery. When incubated with smooth muscle cells that express a biotin receptor, worm micelles specifically bound to the cell surface and transferred their dye contents. As a result, the worm micelles have a demonstrated utility, not only to encapsulate and delivery active agents, but they have proven their potential for ‘targeted’ drug delivery to specific cell types. Moreover, the phase behavior of networks of polymers with differing bending rigidities were simulated using equilibrium statistical mechanical techniques. The insights gained herein also suggest selective chemical modification of the worm micelles would permit the formation of synthetic cytoskeleton-like networks with rich phase behaviors.

All patents, patent applications and publications referred to in the present specification are also fully incorporated by reference.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the basic principles of the invention. Such modifications and additional embodiments are also intended to fall within the scope of the appended claims.

1. A worm-like micelle, capable of encapsulating at least one encapsulant therein, wherein the worm-like micelle comprises one or more wholly synthetic, polymeric, super-amphiphilic molecules that self assemble in aqueous solution, without organic solvent or post assembly polymerization, and wherein at least one of said super-amphiphilic molecules is a hydrophilic block copolymer, the weight fraction (w) of which, relative to total copolymer molecular weight, directs assembly of the amphiphilic molecules into the worm-like micelle of up to one or more microns in length, and determines its stability, flexibility and convective responsiveness.

2. The worm-like micelle of claim 1, wherein the one or more super-amphiphilic molecules of the micelle comprise at least one di-block copolymer.

3. The worm-like micelle of claim 1, wherein all of the super-amphiphilic molecules comprising the micelle are block copolymers.

4. The worm-like micelle of claim 1, wherein the block copolymer comprises >42% hydrophilic polyethylene oxide (PEO) or polyethylene glycol (PEG) and at least one hydrophobic block that drives self-assembly of the worm-like micelle.

5. The worm-like micelle of claim 4, wherein increasing molecular weight of the copolymers increases both core diameter of the worm-like micelle and its stiffness in terms of persistence length.

6. The worm-like micelle of claim 4, wherein at least a portion of the one or more super-amphiphilic molecules of the micelle are chemically cross-linked.

7. The worm-like micelle of claim 1, wherein the at least one encapsulant is selected from the group of active agents consisting of therapeutic compound, dye, indicator, biocide, nutrient, protein or protein fragment, salt, gene or gene fragment, steroid, and gas.

8. The worm-like micelle of claim 1, wherein the worm-like micelle is biocompatible.

9. A method of encapsulating at least one encapsulant into the worm-like micelle of claim 1, wherein the method comprises preparing the worm-like micelle and loading the worm-like micelle by encapsulating therein at least one encapsulant from an environment immediately surrounding the worm-like micelle, thereby removing the at least one encapsulant from said surrounding environment and effecting loading of the worm-like micelle with the at least one encapsulant.

10. The method of encapsulation of claim 9, further comprising controlling encapsulation by destabilizing the loaded worm-like micelle by exposing it and the at least one encapsulant to one or more chemicals or to propagated light, X-ray or UV waves, IR irradiation, sound, ultrasound, heat, or motion.

11. The method of claim 9, wherein the at least one encapsulant is selected from the group of active agents consisting of therapeutic compound, dye, indicator, biocide, nutrient, protein or protein fragment, salt, gene or gene fragment, steroid, and gas.

12. The method of claim 9, further comprising transporting the loaded worm-like micelle and the at least one material encapsulated therein away from the surrounding environment, thereby removing the loaded worm-like micelle and at least one encapsulant from said environment.

13. The method of claim 9, further comprises introducing the worm-like micelle into a patient, so that the environment surrounding the worm-like micelle is within the patient, and effecting encapsulation within the patient; thereby removing the encapsulant from the patient as the loaded worm-like micelle and the at least one encapsulant contained therein are excreted or removed from the patient.

14. The method of claim 13, wherein the worm-like micelle is biocompatible.

15. The method of claim 14, wherein the at least one encapsulatable material is selected from the group consisting of dyes, indicators, biocides, protein or protein fragments, radioactive materials, waste products and unwanted materials.

16. The method of using the worm-like micelle of claim 1 as a delivery vehicle, wherein the method comprises:
preparing the worm-like micelle;
loading the worm-like micelle by encapsulating therein at least one encapsulant;
delivering the loaded worm-like micelle to a selected environment; and
releasing said at least one encapsulant into the environment immediately surrounding the worm-like micelle.

17. The delivery method of claim 16, further comprising delivering the loaded worm-like micelle to a patient, such that the environment surrounding the delivered, loaded worm-like micelle is within the patient, and releasing the encapsulant to the patient.

18. The delivery method of claim 17, wherein the the worm-like micelle is biocompatible.

19. The method of claim 18, wherein the at least one encapsulatable material is selected from the group of active agents consisting of therapeutic compound, dye, indicator, biocide, nutrient, protein or protein fragment, salt, gene or gene fragment, steroid, and gas.

20. The method of claim 18, further comprising controlling release of the at least one encapsulant by destabilizing the loaded worm-like micelle by exposing it and the at least one encapsulant to one or more chemicals or to propagated light, X-ray or UV waves, IR irradiation, sound, ultrasound, heat, or motion.

21. The worm-like micelle of claim 1, which when loaded with at least one encapsulant and transported to a target site, said worm-like micelle comprises a delivery vehicle for delivering the at least one encapsulant to the target.

22. The worm-like micelle of claim 1, which when loaded with at least one encapsulant and administered to a patient, said worm-like micelle comprises a delivery vehicle for delivering the at least one encapsulant to the patient.


SEQUENCE LISTING (1 sequence)

SEQ ID NO: 1 - protein, 12 aa, human transferrin receptor binding peptide
THRPPMWSPVWP